(19) United States
(12) Patent Application Publication
(10) Pub. No.: US 2010/0023091 A1
(43) Pub. Date: Jan. 28, 2010

(54) ACOUSTIC COMMUNICATION OF IMPLANTABLE DEVICE STATUS

(76) Inventors: Jeffrey E. Stahmann, Ramsey, MN (US); Keith R. Maile, New Brighton, MN (US); Paul J. Huelskamp, St. Paul, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/466,097

(22) Filed: May 14, 2009

Related U.S. Application Data
(60) Provisional application No. 61/083,193, filed on Jul. 24, 2008.

(51) Int. Cl. A61N 1/08

(52) U.S. Cl. 607/60

(57) ABSTRACT

An operational status of an implantable medical device is monitored. The implantable medical device includes a biosensor and an acoustic transducer adapted to transmit and receive acoustic signals. An acoustic link is established with the implantable medical device via a remote acoustic transducer adapted to receive acoustic signals from the implantable medical device and to transmit acoustic signals. Data related to the operational status of the implantable medical device is received from the implantable medical device via the acoustic link.

### CROSS-REFERENCE TO RELATED APPLICATION

This application claims priority to Provisional Application No. Jul. 24, 2008, filed 61/083,193, which is herein incorporated by reference in its entirety.

### TECHNICAL FIELD

The present invention relates generally to implantable medical devices. More particularly, the present invention relates to communication of implantable medical device status via an acoustic link.

### BACKGROUND

Implantable medical devices (IMDs) can be placed in the body for monitoring a variety of properties such as temperature, blood pressure, strain, and fluid flow. In some cases, the IMD can be configured to sense other chemical properties, electrical properties, and/or magnetic properties within the body. In addition, implantable medical devices can perform one or more therapeutic functions, such as pacing or defibrillation.

In certain applications, the IMD can be used in conjunction with other devices located inside or outside of a patient's body for performing therapy on the patient. In some applications, for example, an implantable pressure sensor can be used in conjunction with one or more cardiac rhythm management (CRM) devices for predicting the onset of congestive heart failure and delivering an appropriate therapy to a patient. In addition, some implantable sensing devices can also be used for monitoring and treating hypertension, in automatic CRM device settings optimization, and in rhythm discrimination.

Implanting an IMD generally involves delivering and anchoring the IMD at a desired location within the body. However, once anchored in the body, various events can influence the operation of the IMD and the quality of the signal transmitted by the IMD to other implanted devices or an external device. For example, when energy from the IMD battery is depleted, the IMD may no longer be able to perform its designated function or transmit information to other devices. In addition, components of the IMD may malfunction or become damaged after implantation, or the software in the IMD may not execute properly.

### SUMMARY

One aspect of the present invention relates to monitoring an operational status of an implantable medical device. The implantable medical device includes a biosensor and an acoustic transducer adapted to transmit and receive acoustic signals. An acoustic link is established with the implantable medical device via a remote acoustic transducer adapted to receive acoustic signals from the implantable medical device and to transmit acoustic signals. Data related to the operational status of the implantable medical device is received from the implantable medical device via the acoustic link.

In another aspect of the present invention an implantable medical device is maintained after implantation. The implantable medical device includes a biosensor and an acoustic transducer adapted to transmit and receive acoustic signals. An acoustic link is established with the implantable medical device via a remote acoustic transducer adapted to receive acoustic signals from the implantable medical device and to transmit acoustic signals. Data related to an operational status of the implantable medical device is received from the implantable medical device via the acoustic link. The data related to the operational status of the implantable medical device is evaluated to assess whether the implantable medical device is functioning properly. If the data related to the operational status of the implantable medical device indicates that the implantable medical device is not functioning properly, a mitigating acoustic signal is transmitted to the implantable medical device that is configured to resolve an abnormality in the implantable medical device.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a simplified diagram of a network of implantable medical devices implanted in a human body according to an embodiment of the present invention.

FIG. 2 is a functional block diagram illustrating a primary implantable medical device according to an embodiment of the present invention.

FIG. 3 is a functional block diagram illustrating a remote implantable medical device according to an embodiment of the present invention.

FIG. 4 is a functional block diagram illustrating an external device according to an embodiment of the present invention.

FIG. 5 is a flow diagram of a process for communicating device status information from an implantable medical device according to an embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

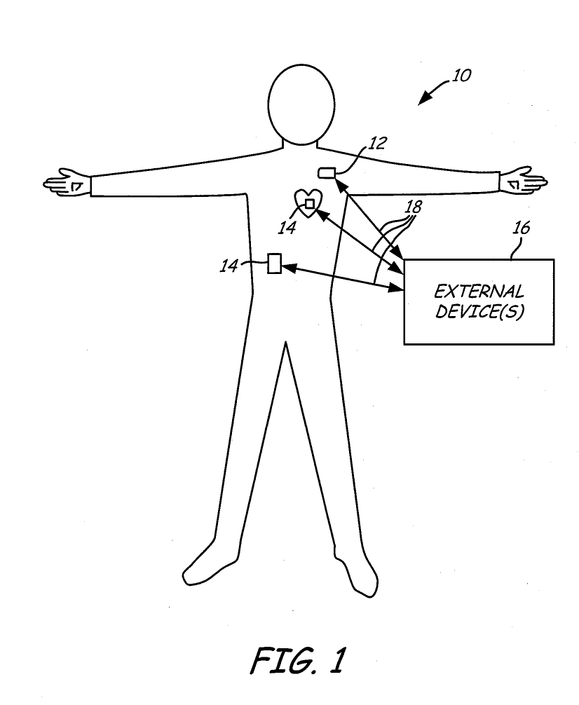

FIG. 1 illustrates a simplified human body in which a system or network 10 of implantable medical devices is implanted. The system 10 includes a primary IMD 12 and at least one remote IMD 14. Although the primary IMD 12 and the remote IMDs 14 are shown implanted in specific locations, in practice, either or both of the primary IMD 12 and the remote IMDs 14 may be implanted anywhere in the body. The system 10 may also include an external device 16 (e.g., a computing device and/or programming device), which may communicate with the primary IMD 12 and/or the remote IMD(s) 14 via communication channels 18. Although FIG. 1 illustrates the system 10 utilizing two remote IMDs 14, those skilled in the art will appreciate that one or more than two remote IMDs 14 may be used within the scope of the present invention.

Each remote IMD 14 may be configured to perform one or more designated functions, which may include taking one or more physiological measurements and/or delivering a desired therapy. The implantation sites for the remote IMD 14 are determined based on the particular therapeutic needs of the patient. In one embodiment, the remote IMD 14 is adapted to be implanted to measure blood pressure within the patient's pulmonary artery, and to store and/or transmit blood pressure data to the primary IMD 12, another IMD, or external device(s) 16. Other types of physiological parameters that the remote IMD 14 may be configured to measure include temperature, blood gas content, strain, fluid flow, chemical properties, electrical properties, and magnetic properties. In another embodiment, the remote IMD 14 is adapted to deliver a desired therapy (e.g., a pacing and/or defibrillation stimulus) to the patient's heart or cardiovascular system. In yet another embodiment, the remote IMD 14 is adapted to measure a non-physiological parameter that is affected by, or may affect, a patient or one or more of a patient's IMDs. Examples of non-physiological parameters include, for example, electromagnetic energy, ionizing radiation, barometric pressure, and geographic location.

The remote IMD 14 includes power supply components (e.g., a battery) for providing electrical power to the various components and/or circuitry for performing the functions described above. The remote IMD 14 is desirably made as small as possible, however, which constrains the space within the remote IMD 14 that is available for power supply components. Such space constraints limit the capacity of these power supply components. In an effort to maximize the longevity of the remote IMD 14, its power consumption is minimized, and thus, the average power consumption of the remote IMD 14 is desirably very low.

In order to achieve this low power consumption, the remote IMD 14 is normally in a “sleep” or “sleeping” state (i.e., an inactive state) characterized by a power consumption of from essentially zero (i.e., a completely powered off state) to a low power state in which only a minimal circuitry (e.g., a timer or comparator) are energized and consuming electrical power. The remote IMD 14 is awakened (i.e., powered on) to an active state in which it can perform one or more designated functions. The terms “wake,” “waking,” “wake-up,” and “awaken” relate to the operation of powering on or energizing one or more aspects of the remote IMD 14 to an active state, such that the awakened portion can perform a designated function.

The remote IMD 14 may be awakened by, for example, the primary IMD 12 or the external device 16. The remote IMD 14 is desirably in the active state only to the extent necessary to perform its designated diagnostic and/or therapeutic function(s), after which time it returns to its inactive, sleep state. Additionally, in some embodiments, to maximize the longevity of the remote sensor IMD 14, the sleep state is minimized such that the ratio of the sleep current to wake current is less than 10%. In some embodiments, the remote IMD 14 is configured to wirelessly communicate with the primary IMD 12, the other remote IMD 14, and/or the external device(s) 16 in the active state by transmitting a single acoustical pulse or series of pulses.

The primary IMD 12 operates, in some embodiments, to wake the remote IMD 14 from the sleep state, and may further be configured to direct the remote IMD 14 to perform one or more designated functions. In this way, the primary IMD 12 functions as a “master” device while the remote IMD 14 functions as a “slave” device. The primary IMD 12 itself may also be configured to perform therapeutic functions or to take physiologic measurements. For example, the primary IMD 12 may, in some embodiments, be a pulse generator for providing a cardiac pacing and/or defibrillation stimulus. The therapeutic functions are not limited to any particular type and can include, for example, drug delivery therapy, or any other therapy capable of being administered with an IMD. Additionally, the primary IMD 12 may be configured to measure physiologic parameters such as blood pressure, temperature, blood or fluid flow, strain, electrical, chemical, or magnetic properties within the body.

It should be noted that neither the remote IMD 14 nor the primary IMD 12 are limited to any particular type or types of devices. For example, the remote IMD 14 can be any IMD that is normally in a sleep state to minimize power consumption and is awakened only as necessary to perform a desired function. Similarly, the primary IMD 12 can be any IMD that operates, at least in part, to cause the remote IMD 14 to wake from a sleep state. Thus, in this regard, the remote IMD 14 may sometimes also function as the primary IMD 12 in a given embodiment. That is, the remote IMD 14 may be configured such that, in its active state, it can cause another remote IMD 14 to wake and perform one or more desired functions.

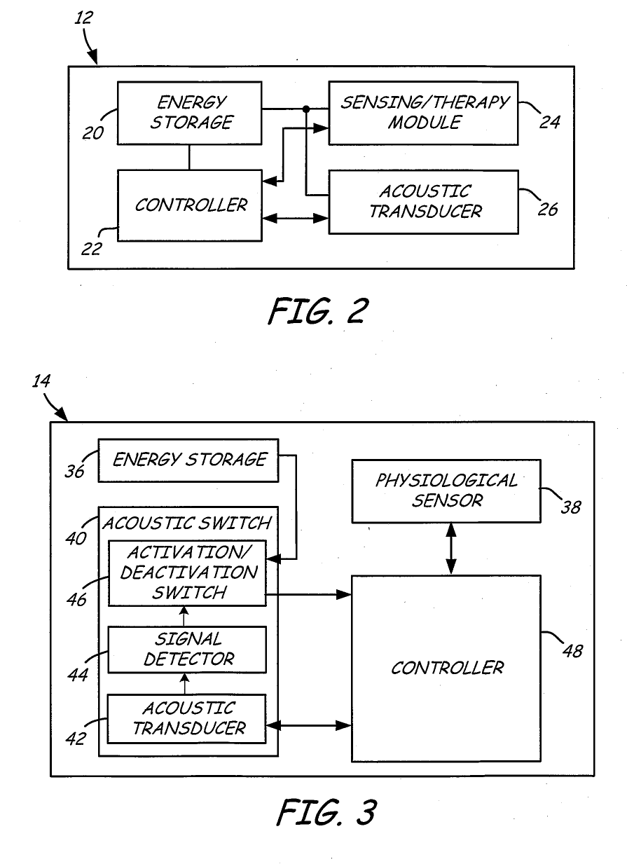

FIG. 2 is a functional block diagram illustrating an embodiment of the primary IMD 12. The primary IMD 12 includes an energy storage device 20, a primary IMD controller 22, a sensing and/or therapy module 24, and an acoustic transducer 26. In some embodiments, the primary IMD 12 may not include the sensing and/or therapy module 24. The term “module” is not intended to imply any particular structure. Rather, “module” may mean components and circuitry integrated into a single unit as well as individual, discrete components and circuitry that are functionally related.

The energy storage device 20 operates to provide operating power to the controller 22, the sensing and/or therapy module 24, and the acoustic transducer 26. The controller 22 operates to control the sensing and/or therapy module 24 and the acoustic transducer 26, each of which is operatively coupled to and communicates with the controller 22. For example, the controller 22 may command the sensing and/or therapy module 24 to deliver a desired therapy, such as a pacing or defibrillation stimulus. In addition, the controller 22 may command the acoustic transducer 26 to transmit and/or receive data from the external device 16 or the remote IMDs 14.

The primary IMD 12 may also include timing circuitry (not shown) which operates to schedule, prompt, and/or activate the primary IMD 12 to perform various activities. For example, in one embodiment, the timing circuitry may be utilized to determine the appropriate time at which one or more remote IMDs 14 should wake in order to perform a designated function. In one embodiment, the timing circuitry may be an internal timer or oscillator, while in other embodiments, timing may be performed by specific hardware components that contain hardwired logic for performing the steps, or by any combination of programmed computer components and custom hardware components.

The acoustic transducer 26 is configured to both transmit and receive acoustic signals to and from other devices, such as the external device 16 or the remote IMD 14. In other embodiments, the primary IMD 12 includes at least one transducer configured for receiving an acoustic signal and at least one transducer for transmitting an acoustic signal. The acoustic transducer 26 generates an electrical signal proportional to the magnitude of acoustic energy received by the transducer 26, which is then conveyed to the controller 22. In similar fashion, the acoustic transducer 26 generates an acoustic signal proportional to the magnitude of the electrical energy generated by the controller 22. An example acoustic transducer that can be used in small profile external units is disclosed in U.S. patent application Ser. No. 11/287,557, entitled “Implantable Medical Device with Integrated Acoustic Transducer,” which is expressly incorporated herein by reference in its entirety.

The sensing and/or therapy module 24, if present, operates to perform the therapeutic and/or diagnostic functions described above. In one embodiment, the sensing and/or therapy module 24 delivers a cardiac pacing and/or defibrillation stimulus. Again, the sensing and/or therapy module 24 is not limited to performing any particular type of physiologic measurement or therapy.

FIG. 3 is a functional block diagram illustrating an embodiment of the remote IMD 14. The remote IMD 14 includes an energy storage device 36, a physiological sensor 38, an acoustic switch 40 (including an acoustic transducer 42, a signal detector 44, and an activation/deactivation switch 46), and a remote IMD controller 48. The energy storage device 36 may be non-rechargeable or rechargeable. The energy storage device 36 operates to supply power to the physiological sensor 38, the acoustic switch 40, and controller 48.

The controller 48 may include a microprocessor or microcontroller coupled to a memory device that includes operating instructions and/or software for the microprocessor or microcontroller. The remote IMD 12, and in particular the controller 48, may also include timing circuitry which operates to direct the activities of the remote IMD 14 (e.g., taking and storing physiologic measurements, uploading measurement data) after it has been awakened from its sleep state. Alternatively, the remote IMD controller 48 may have reduced functionality as compared to the primary IMD controller 22, in embodiments where the functional requirements of the remote IMD 14 are less extensive.

The physiological sensor 38 performs functions related to measurement of physiological parameters, and is not limited to any particular type of physiological measurement. For example, the physiological sensor 38 may be a pressure sensor adapted to measure internal pressure in a blood vessel. In one such embodiment, the remote IMD 14 is implanted in the patient's pulmonary artery, and the physiological sensor 38 is adapted to measure blood pressure therein. An example remote IMD 14 operable to measure blood pressure, which is suitable for use in conjunction with the present invention, is disclosed in U.S. patent application Ser. No. ______, entitled “Implantable Pressure Sensor with Automatic Measurement and Storage Capabilities,” which is hereby incorporated by reference in its entirety. In other embodiments, physiological sensor 40 is adapted to generate a signal related to other sensed physiological parameters including, but not limited to, temperature, electrical impedance, position, strain, pH, blood flow, radiation level, and glucose level.

Remote IMD 14 may also have the capability to perform one or more therapeutic functions (e.g., cardiac pacing, drug delivery) in addition to, or in lieu of, one or more measurement functions. In one such embodiment, remote IMD 14 includes a therapy delivery module and does not include physiological sensor 40.

The acoustic transducer 42 may include one or more piezoelectric transducer elements configured for transmitting and receiving acoustic signals. In a reception mode of operation, the acoustic transducer 42 generates an electrical signal proportional to the magnitude of the acoustic signal wirelessly received from the primary IMD 12 or the external device 16, which is then conveyed to the controller 48 when the remote IMD 14 is in the active state. Similarly, in a transmission mode of operation the acoustic transducer 42 generates an acoustic signal proportional to the magnitude of the electrical signal conveyed from the controller 48 when the remote IMD 14 is in the active state, which is then wirelessly transmitted to the primary IMD 12 or the external device 16.

The signal detector 44 is configured to generate an activation trigger signal to activate the remote IMD 14 via the activation/deactivation switch component 46. The activation trigger signal is generated by the signal detector 44 when the electrical signal generated by the acoustic transducer 42 exceeds a specific voltage threshold. The activation/deactivation switch component 46 is the component through which current is delivered from the energy storage device 36 to the controller 48 when actuated. In response to the generation of the activation trigger signal by the signal detector 44, the switch component 46 is actuated to allow current to flow to the controller 48, thereby placing the remote IMD 14 in the active state. The switch component 46 can also be actuated to prevent current from flowing to the controller 48, thereby placing the remote IMD 14 in the standby state. Further details regarding the general construction and function of acoustic switches are disclosed in U.S. Pat. No. 6,628,989, entitled “Acoustic Switch And Apparatus And Methods For Using Acoustic Switches Within The Body,” which is hereby incorporated by reference in its entirety. In other embodiments, the primary IMD 12 or the external device 16 operates to generate a field (i.e., a wake-up field) that can be detected by a sensing module in the remote IMD 14 for the purpose of causing the remote IMD 14 to wake from the sleep state.

As discussed previously, an acoustical activation or wake-up signal can be used to activate the remote IMD 14 when the remote IMD 14 is in the standby state. When in the standby state, the electrical signal is not passed to the controller 48, but rather acts solely to close the activation/deactivation switch 46. To activate the remote IMD 14, one or more activation acoustic energy waves or signals can be transmitted from the primary IMD 12 or the external device 16 into the patient's body towards the remote IMD 14, which is received by the acoustic transducer 42. Upon excitation, the acoustic transducer 42 generates an electrical signal that causes the signal detector 44 to generate a trigger signal that is used to close, open, or otherwise activate the activation/deactivation switch 46. In some embodiments, physiological sensor 38, acoustic switch 40, and controller 48 may be integrated into an integrated circuit, while in other embodiments one or more of these elements may be discrete hardware and circuitry.

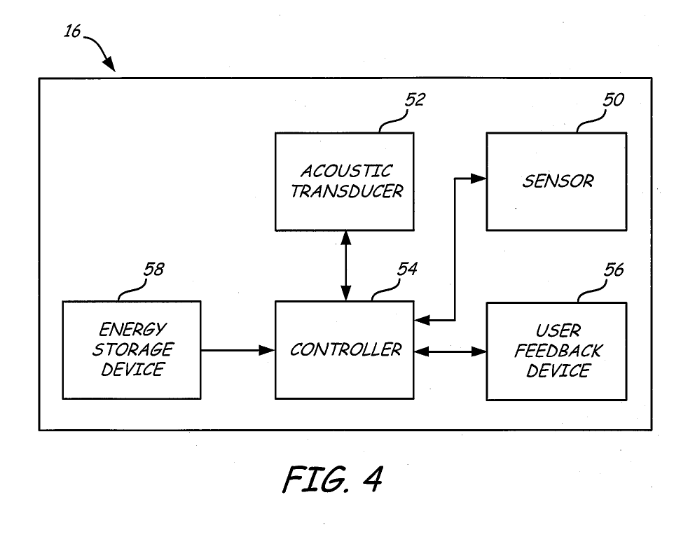

FIG. 4 is a functional block diagram illustrating an embodiment of the external device 16. The external device 16 includes an on-board sensor 50, an acoustic transducer 52, a controller 54, an audio/visual user feedback device 56, and an energy storage device 58. In some embodiments, external device 16 is a handheld device for use by a caregiver for acoustically communicating with the primary IMD 12 and/or the remote IMD 14.

The sensor 50 may comprise a biosensor that generates a signal in response to a measured parameter. In one embodiment, the sensor 50 comprises a barometric pressure sensor configured measure barometric pressure for use in calibrating the remote IMD 14. The external device 16 may include one or more additional sensors such as an ECG electrode sensor, a systemic blood pressure sensor, a posture sensor, a global positioning sensor (GPS), an activity sensor, a temperature sensor, a timer, and/or an oximeter.

The acoustic transducer 52 for the external device 16 is configured to both transmit and receive acoustic signals to and from the primary IMD 12 and/or the remote IMD 14. In other embodiments, the external device 16 includes at least one transducer configured to receive an acoustic signal and at least one transducer for transmitting an acoustic signal. The acoustic transducer 52 generates an electrical signal proportional to the magnitude of acoustic energy received by the transducer 52, which is then conveyed to the controller 54. In a similar manner, the acoustic transducer 52 generates an acoustic signal proportional to the magnitude of the electrical energy generated by the controller 54.

The controller 54 includes circuitry for activating or controlling the sensor 50 and for receiving signals from the sensor 50. In some embodiments, the controller 54 may include an oscillator or other circuitry for wirelessly transmitting acoustic signals to the primary IMD 12 and/or the remote IMD 14 via the acoustic transducer 52. The controller 54 can also include signal detection circuitry in some embodiments for wirelessly receiving acoustic signals from the primary IMD 12 and/or the remote IMD 14 via the acoustic transducer 52 or from another acoustic transducer coupled to the external device 16.

In some embodiments, the controller 54 includes a processor for analyzing, interpreting, and/or processing the received acoustic signals, and a memory for storing the processed information and/or commands for use internally. In certain embodiments, for example, the controller 54 can be used to analyze the strength and quality of the acoustic signal received from the IMD 12. The controller 54 can be configured as a digital signal processor (DSP), a field programmable gate array (FPGA), an application specific integrated circuit (ASIC)-compatible device such as a CoolRISC processor available from Xemics or other programmable devices, and/or any other hardware components or software modules for processing, analyzing, storing data, and controlling the operation of the external device 16.

The user feedback device 56 can include a screen or display panel for communicating information to the clinician and/or to the patient. For example, the screen or display panel may be configured to display operational or diagnostic information about the primary IMD 12 and/or the remote IMD 14. As another example, the screen or display panel can display visual information indicative of the strength and/or quality of the acoustic signal received from each remote IMD 14 for use in assessing whether a target region within the body is acceptable for providing a sufficient acoustic link between the remote IMD 14 and another implant (e.g., the primary IMD 12) and/or external device in acoustic communication with the remote IMD 14. In certain embodiments, where the external device 16 is integrated into another device, the screen or display panel may also be used to display other information such as any physiological parameters monitored by the remote IMD 14.

In some embodiments, the external device 16 can include an interface for connecting to the Internet, to a cell phone, and/or to other wired or wireless means for downloading or uploading information and programs, debugging data, and upgrades. In some embodiments, this connection may also be used for charging the energy storage device 58 within the external device 16. According to some embodiments, the external device 16 may also be capable of operating in two modes: a user mode that provides useful clinical information to the patient or a caregiver, and a diagnostic mode that provides information to an individual for calibrating and/or servicing the external device 16.

To assess whether acoustic communication between the remote IMD 14 and the primary IMD 12 and/or the external device 16 is adequate, the primary IMD 12 or external device 16 transmits an acoustic signal to the remote IMD 14. Upon receiving the acoustic signal, the remote IMD 14 enters into a transmission mode and transmits an acoustic signal back to the primary IMD 12 or external device 16. The primary IMD 12 or external device 16 evaluates the strength and quality of the acoustic signal received from the remote IMD 14. When the external device 16 evaluates the acoustic signal strength and quality, information about the strength and quality of the acoustic signal may be provided to the clinician via the user feedback device 56 as discussed with respect to FIG. 4.

The external device 16 may individually evaluate and display the acoustic signal strength and quality of multiple acoustic communication paths. For example, external device 16 may individually evaluate and display the acoustic signal strength of the communication path, wherein the external device 16 is the receiver and the primary IMD 12 is the transmitter. Further the external device 16 may individually evaluate and display the acoustic signal strength of the communication path wherein the external device 16 is the transmitter and the primary IMD 12 is the receiver. Any other communication path between the external device 16, the primary IMD 12, and one or more of the remote IMDs 14 may also be evaluated and displayed.

When the remote IMD 14 is implanted in a patient, it is important to monitor the operational status of the remote IMD 14. For example, the quality and strength of the acoustic link, the status of the physiological sensor 38, the operation of the software stored and run by the controller 48, and the remaining energy of the energy storage device 36 are all operational factors of the remote IMD 14 that may have an effect on the therapy and/or sensing capabilities of the remote IMD 14. By assuring that the remote IMD 14 is functioning properly, therapy and/or sensing processes provided by the remote IMD 14 can continue uninterrupted, thereby assuring consistent treatment of the condition monitored by the remote IMD 14.

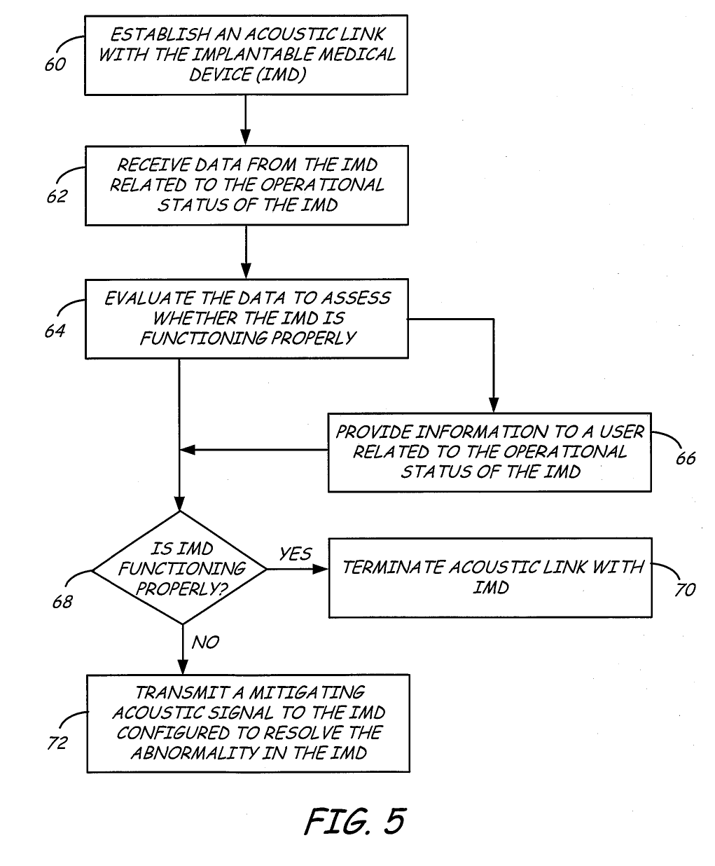

The remote IMD 14 according to the present invention is configured to acoustically communicate operational status information to other devices or systems, such as the primary IMD 12 or the external device 16. FIG. 5 is a flow diagram of a process for communicating device status information from the remote IMD 14 according to an embodiment of the present invention. In step 60, an acoustic link is established between the remote IMD 14 and either of the primary IMD 12 and the external device 16. The communication link may be established as described above, with the primary IMD 12 or the external device 16 sending an acoustic signal to the remote IMD 14 to wake up the remote IMD 14. For example, the communication link may be established at an appointment with a caregiver. The remote IMD 14 then sends an acoustic signal back in response to establish the acoustic link between the devices. In this case, the primary IMD 12 and/or the external device(s) 16 is configured to “pull” the device status from the remote IMD 14.

In an alternative embodiment, the remote IMD 14 wakes up automatically, based on, for example, a change in the status of a component in the remote IMD 14, or a schedule programmed into controller 38 of the remote IMD 14. The remote IMD 14 then sends an acoustic signal to either of the primary IMD 12 or the external device 16 to establish the acoustic link between the devices. In this case, the remote IMD 14 is configured to “push” the device status information to the primary IMD 12 and/or the external device(s) 16.

In step 62, the device linked to the remote IMD 14 (e.g., the primary IMD 12 or the external device 16) receives data from the remote IMD 14 related to the operational status of the remote IMD 14. The data related to the operational status of the remote IMD 14 may include at least one of information regarding the status of energy storage device 36, component status information (e.g., memory status, status of acoustic transducer 42), detected error information (e.g., software or hardware error), operational mode change information (e.g., a transition from normal to fault mode operation), communication error information, corrected software error information, corrected hardware error information, communication quality information (e.g., signal-to-noise ratio, number of communication retries), biosensor status information (e.g., sensor drift, gain, test signal), and oscillator status information. This list is non-exclusive, and any type of information that is related to the operation of the remote IMD 14 can be communicated by the remote IMD 14. Communication of the operational status of the remote IMD 14 is important to the safety of the overall network of devices shown in FIG. 1, the effectiveness of the network in adapting faults and errors in the remote IMD 14, convenience of knowing the remaining life of the energy storage device 36, and patient assurance that the remote IMD 14 is operating properly.

If the data from the remote IMD 14 is received by the primary IMD 12, the data may be processed by the controller 22 or may be stored in internal memory of the controller 22. In step 64, the primary IMD 12 may evaluate the data received from the remote IMD 14 to assess whether the remote IMD 14 is functioning properly. The primary IMD 12 may also act as the master to a plurality of remote IMDs 14 to collect and store the operational status data from the plurality of remote IMDs 14. The operational status data for the remote IMDs 14 may then be transmitted from the primary IMD 12 to another device (e.g., the external device 16) with one connection, thereby minimizing the draw on the energy storage device 20 of the primary IMD 12.

If the operational status data from the remote IMD 14 is received by the external device 16, the data may be processed by controller 54 or may be stored in internal memory of the controller 54. In step 64, the external device 16 may evaluate the data received from the remote IMD 14 to assess whether the remote IMD 14 is functioning properly. Then, in optional step 66, the processed data may also be provided to a user of the external device 16 (e.g., a caregiver) for review. For example, the controller 54 may process the raw data from the remote IMD 14 and display the information on the user feedback device 56. The information displayed on the user feedback device 56 may be in the form of a table or list of the operational parameters. The display may list all operational parameters tested by the remote IMD 14, with a notation of which parameters included abnormalities. Alternatively, the display may list only those operational parameters that included abnormalities. In any case, the user may then determine whether additional remedial steps to correct abnormalities in the operation of the remote IMD 14 should be taken.

In decision step 68, if the primary IMD 12 or the external device 16 determines that the remote IMD 14 is functioning properly based on the received operational status information, the acoustic link with the remote IMD 14 is terminated in step 70. The primary IMD 12 or the external device 16 may transmit a signal to the remote IMD 14 to terminate the acoustic link. Alternatively, the remote IMD 14 may be programmed to automatically terminate the acoustic link after transmission of the operational status data.

If, in decision step 68, the primary IMD 12 or the external device 16 determines that the remote IMD 14 is not functioning properly, the primary IMD 12 or the external device 16 may transmit a mitigating acoustic signal to the remote IMD 14 in step 72. The mitigating signal is designed to resolve the abnormality found in the operation of the remote IMD 14, and may be sent automatically by the external device 16 or in response to a request by the user of external device 16. For example, if the operational status data indicates that one of the hardware components of the remote IMD 14 failed and resulted in an error or fault, the primary IMD 12 or the external device 16 may send a reset signal to the remote IMD 14 to reset or reboot the components of the remote IMD 14. As another example, if the operational status data indicates that an error or fault has occurred in the software run by the controller 48, the primary IMD 12 or the external device 16 may send a new version of the software or a patch to the remote IMD 14 to rectify the software issue. As a further example, if the operational status data indicates that the available energy in energy storage device 36 is low, the primary IMD 12 or the external device 16 may be configured to provide an electromagnetic or acoustic charging signal to the remote IMD 14 to recharge the energy storage device 36. After the mitigating signal is received by the remote IMD 14, the acoustic link may be terminated in step 70.

In summary, the present invention relates to monitoring an operational status of an implantable medical device. The implantable medical device includes a biosensor and an acoustic transducer adapted to transmit and receive acoustic signals. An acoustic link is established with the implantable medical device via a remote acoustic transducer adapted to receive acoustic signals from the implantable medical device and to transmit acoustic signals. Data related to the operational status of the implantable medical device is received from the implantable medical device via the acoustic link. For example, monitoring the operational status of the remote may assure that the remote implantable medical device is capable of transmitting physiological signals measured by the physiological sensor, and that the physiological signals are not corrupted. This allows therapy and/or sensing processes provided by the remote implantable medical device to continue uninterrupted, thereby assuring consistent treatment of the condition monitored by the remote implantable medical device.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof. For example, while the communication of operational status information has been described with regard to an implantable cardiac device, the principles of the present invention are also applicable to other types of chronically implanted medical devices.

We claim:

1. A method for monitoring an operational status of an implantable medical device, wherein the implantable medical device includes a biosensor and an acoustic transducer adapted to transmit and receive acoustic signals, the method comprising:
establishing an acoustic link with the implantable medical device via a remote acoustic transducer adapted to receive acoustic signals from and transmit signals to the implantable medical device; and
receiving data from the implantable medical device related to the operational status of the implantable medical device via the acoustic link.

2. The method of claim 1, and further comprising:
providing information to a user related to the operational status of the implantable medical device.

3. The method of claim 2, wherein providing information to the user comprises displaying information related to the operational status of the implantable medical device on a display.

4. The method of claim 1, wherein establishing an acoustic link with the implantable medical device comprises responding to the implantable medical device when the remote acoustic transducer transmits a link initiating signal.

5. The method of claim 1, and further comprising:
evaluating the data related to the operational status of the implantable medical device to assess whether the implantable medical device is functioning properly.

6. The method of claim 5, and further comprising:
transmitting a mitigating acoustic signal to the implantable medical device if the data related to the operational status of the implantable medical device indicates that the implantable medical device is not functioning properly, wherein the mitigating acoustic signal is configured to resolve an abnormality in the implantable medical device.

7. The method of claim 1, wherein the information related to the operational status of the implantable medical device includes at least one of component status information, battery status information, detected error information, operational mode change information, communication error information, corrected software error information, corrected hardware error information, communication signal strength and quality information, and biosensor status information.

8. The method of claim 7, wherein the information related to the operational status is provided to a user and the information is specific to at least one of the implantable medical device and a communication path between the implantable medical device and another implantable medical device.

9. A system comprising:
an implantable medical device including a biosensor and an acoustic transducer adapted to transmit and receive acoustic signals;
a processing device including an acoustic transducer adapted to receive acoustic signals from the implantable medical device and to transmit acoustic signals, wherein the processing device is configured to establish an acoustic link with the implantable medical device and receive data from the implantable medical device related to the operational status of the implantable medical device via the acoustic link.

10. The system of claim 9, wherein the processing device is further configured to evaluate the data related to the operational status of the implantable medical device to assess whether the implantable medical device is functioning properly.

11. The system of claim 9, wherein the processing device is further configured to transmit a mitigating acoustic signal to the implantable medical device if the data related to the operational status of the implantable medical device indicates that the implantable medical device is not functioning properly, wherein the mitigating acoustic signal is configured to resolve an abnormality in the implantable medical device.

12. The system of claim 9, wherein the processing device is an external device.

13. The system of claim 12, wherein the processing device comprises a display.

14. The system of claim 13, wherein the external device is configured to provide information related to the operational status of the implantable medical device on the display.

15. The system of claim 9, wherein the processing device is an implantable device.

16. The system of claim 9, wherein the implantable device comprises a pulse generator.

17. The system of claim 9, wherein the information related to the operational status of the implantable medical device includes at least one of component status information, battery status information, detected error information, operational mode change information, communication error information, corrected software error information, corrected hardware error information, communication signal strength and quality information, and biosensor status information.

18. A method for maintaining an implantable medical device after implantation, wherein the implantable medical device includes a biosensor and an acoustic transducer adapted to transmit and receive acoustic signals, the method comprising:
establishing an acoustic link with the implantable medical device via a remote acoustic transducer adapted to receive acoustic signals from the implantable medical device and to transmit acoustic signals;
receiving data from the implantable medical device related to an operational status of the implantable medical device via the acoustic link;
evaluating the data related to the operational status of the implantable medical device to assess whether the implantable medical device is functioning properly; and
transmitting a mitigating acoustic signal to the implantable medical device if the data related to the operational status of the implantable medical device indicates that the implantable medical device is not functioning properly, wherein the mitigating acoustic signal is configured to resolve an abnormality in the implantable medical device.

19. The method of claim 18, wherein, after receiving data related to the operational status of the implantable medical device, the method further comprises:
providing information to a user related to the operational status of the implantable medical device.

20. The method of claim 19, wherein providing information to the user comprises displaying information related to the operational status of the implantable medical device on a display.
